(19) United States
(12) Patent Application Publication
(10) Pub. No.: US 2025/0072672 A1
(43) Pub. Date: Mar. 6, 2025

(54) TOILET-SEAT DEVICE

(71) Applicant: TOTO LTD., Kitakyushu-shi (JP)

(72) Inventors: Kosuke Goto, Fukuoka (JP); Yusuke Motoda, Fukuoka (JP); Hiroki Hata, Fukuoka (JP); Takeshi Yasugata, Fukuoka (JP)

(21) Appl. No.: 18/786,675

(22) Filed: Jul. 29, 2024

(30) Foreign Application Priority Data
Aug. 31, 2023 (JP) 2023-140835
Aug. 31, 2023 (JP) 2023-140836

(51) Int. Cl. A47K 13/24

(52) U.S. Cl. CPC A47K 13/24

(57) ABSTRACT

A toilet-seat device according to an embodiment includes a hollow toilet seat and a sensor unit. The sensor unit includes a sensor case that is fixed to be exposed from an opening formed in the toilet seat, the sensor case housing therein a sensor substrate. The sensor case includes an engaging part that is engaged with the toilet seat to fix the sensor case to the toilet seat. The engaging part is arranged in a position that is inner than an outer edge of the sensor case.

### CROSS-REFERENCE TO RELATED APPLICATIONS

This application is based upon and claims the benefit of priority of the prior Japanese Patent Application No. 2023-140835, filed on Aug. 31, 2023 and Japanese Patent Application No. 2023-140836, filed on Aug. 31, 2023, the entire contents of which are incorporated herein by reference.

### FIELD

An embodiment of the disclosure relates to a toilet-seat device.

### BACKGROUND

Conventionally, there has been proposed a technology for providing an electrostatic sensor to an inner surface of a toilet seat so as to detect sitting of a user on the basis of an output from the electrostatic sensor (see Japanese Laid-open Patent Publication No. 2019-024933, for example).

Incidentally, among sensor units each including a sensor provided in a toilet seat, some of them are fixed such that a part thereof is exposed from an opening of a toilet seat. In this case, in a case where a body weight of a user that is sitting on a toilet seat is applied to deform the toilet seat, there presents possibility that load works on a sensor substrate in the sensor unit due to deformation of the toilet seat. As described above, in the conventional technology, there has been a room for improvement in reducing load that works on a sensor substrate in a case where a toilet seat is deformed.

### SUMMARY

A toilet-seat device according to one aspect of an embodiment includes: a hollow toilet seat; and a sensor unit including a sensor case that is fixed to be exposed from an opening formed in the toilet seat, the sensor case housing therein a sensor substrate, wherein the sensor case includes an engaging part that is engaged with the toilet seat to fix the sensor case to the toilet seat, and the engaging part is arranged in a position that is inner than an outer edge of the sensor case.

### BRIEF DESCRIPTION OF DRAWINGS

A more complete appreciation of the invention and many of the attendant advantages thereof will be readily obtained as the same becomes better understood by reference to the following detailed description when considered in connection with the accompanying drawings, wherein:

FIG. 1 is a perspective view illustrating a flush toilet device that includes a toilet-seat device according to an embodiment;

FIG. 2 is a block diagram illustrating a configuration example of a measurement system;

FIG. 3 is a perspective view illustrating a sensor unit;

FIG. 4 is an exploded perspective view illustrating the sensor unit;

FIG. 5 is a perspective view illustrating the toilet seat when viewed from the underneath;

FIG. 6 is a cross sectional view taken along a line VI-VI illustrated in FIG. 1; and

FIG. 7 is a diagram illustrating a state where the toilet seat is deformed due to sitting of a user.

### DESCRIPTION OF EMBODIMENTS

Hereinafter, embodiments of a toilet-seat device disclosed in the present application will be described in detail with reference to the accompanying drawings. This invention is not limited to embodiments described below.

### Embodiment

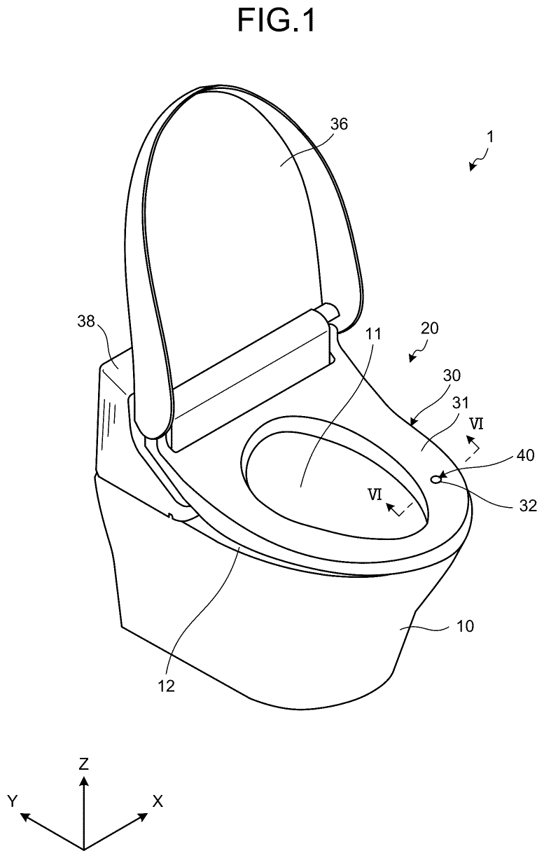

A toilet-seat device according to an embodiment will be explained with reference to FIG. 1. FIG. 1 is a perspective view illustrating a flush toilet device that includes the toilet-seat device according to the embodiment. Note that FIG. 1 and FIG. 3 and the following are schematic diagrams.

In FIG. 1 and the like, for convenience of explanation, a three-dimensional orthogonal coordinate system is illustrated in which an X-axis direction, a Y-axis direction, and a Z-axis direction, which are perpendicular to one another, are prescribed, and the Z-axis positive direction is the upward vertical direction. Hereinafter, an X-axis positive direction, an X-axis negative direction, a Y-axis positive direction, a Y-axis negative direction may be referred to as “left direction”, “right direction”, “backward direction”, and “forward direction”, respectively. In other words, directions viewed from a user sitting on a toilet seat 30 to be mentioned later may be referred to as “left direction”, “right direction”, “backward direction”, and “forward direction”. Thus, in the following explanation, the X-axis direction, the Y-axis direction, and the Z-axis direction may be referred to as a left-right direction, a front-back direction, and an up-and-down direction (vertical direction), respectively.

As illustrated in FIG. 1, a flush toilet device 1 includes a flush toilet 10 and a toilet-seat device 20. The flush toilet 10 includes a bowl part 11. The bowl part 11 is formed in bowl-shaped to be capable of receiving wastes. Flush water is spouted to wash the bowl part 11, and the flush water having washed the bowl part 11 is discharged through a not-illustrated a discharging passage. The flush toilet 10 is made of pottery, for example. Note that the flush toilet 10 is not limited to be made of pottery, and may be made of resin or combination of pottery and resin, for example.

The toilet-seat device 20 is attached to an upper part of the flush toilet 10, and further includes the toilet seat 30, a toilet lid 36, a functional unit 38, and a sensor unit 40. Note that the toilet-seat device 20 may be detachably attached to the flush toilet 10, or may be integrally attached to the flush toilet 10.

The toilet seat 30 is formed in circular-shaped having an opening at the center thereof, and further is arranged in a position to be overlapped with an opening of the flush toilet 10 along a rim part 12. The toilet seat 30 is made of resin, for example, and further includes a seating surface 31 on which a user sit. The seating surface 31 supports a gluteal region and/or a femoral region of a sitting user. The toilet seat 30 is hollowly formed, and the sensor unit 40 is arranged in an inner part thereof.

The toilet lid 36 is attached to the toilet seat 30 to be openable and closable. The functional unit 38 houses therein a nozzle device, for example, and further causes the nozzle device to eject washing water toward a body of a user so as to wash his/her private part. Note that the toilet-seat device 20 may have a configuration without the above-mentioned toilet lid 36 and the functional unit 38.

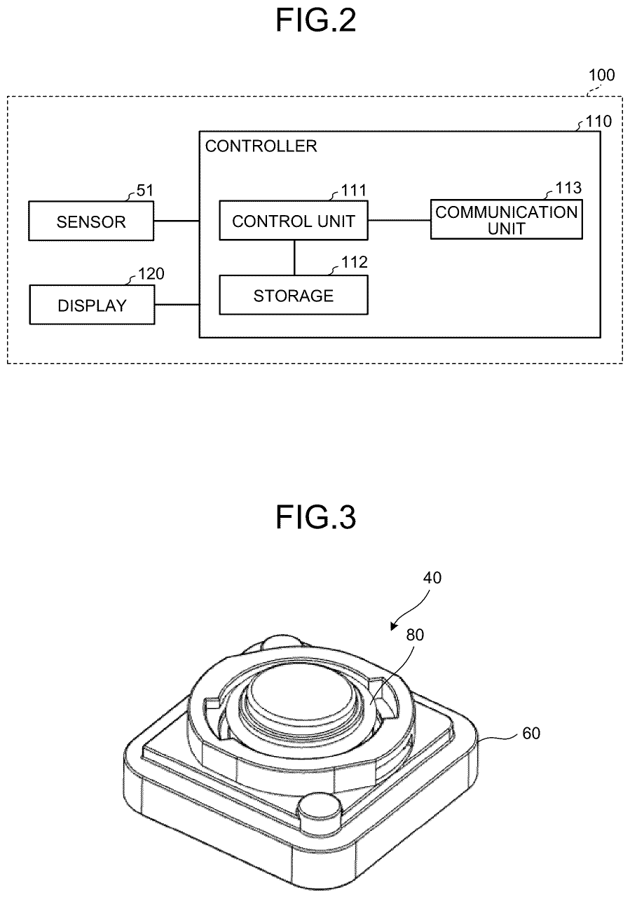

The sensor unit 40 includes a sensor 51 (see FIG. 2) that is capable of measuring various states related to a user using the flush toilet device 1. For example, the sensor 51 is a sensor that is capable of measuring a blood flow state of a user sitting on the toilet seat 30. As the above-mentioned sensor 51, an optical biosensor may be used. Herein, a measurement system including the sensor 51 will be explained with reference to FIG. 2. FIG. 2 is a block diagram illustrating a configuration example of the measurement system.

As illustrated in FIG. 2, a measurement system 100 includes the above-mentioned sensor 51, a controller 110, and a display 120. For example, the sensor 51 irradiates infrared ray, visible light, and/or laser toward a user sitting on the toilet seat 30. Specifically, in a state where a user is sitting on the toilet seat 30, the sensor 51 irradiates infrared ray and the like toward a measurement part in a gluteal region or a femoral region of a user. The sensor 51 causes a blood vessel (blood flow) of a user to reflect irradiated light so as to receive the reflected light (scattering light). The sensor 51 outputs, to the controller 110, a signal related to irradiated light and/or reflected light such as a frequency of the irradiated light and/or the reflected light, for example.

The controller 110 includes a control unit 111, a storage 112, and a communication unit 113. Note that the controller 110 is housed in the functional unit 38 (see FIG. 1) of the toilet-seat device 20, for example; however, not limited thereto.

For example, a Central Processing Unit (CPU), a Micro Processing Unit (MPU), or the like executes a program stored in the storage 112 by using a Random Access Memory (RAM) as a work region so as to realize the control unit 111. Note that the control unit 111 may be realized by hardware such as an Application Specific Integrated Circuit (ASIC) and a Field Programmable Gate Array (FPGA). The storage 112 includes a volatile memory and/or a non-volatile memory, for example, and is realized by a semiconductor memory element such as a RAM and Flash Memory.

On the basis of a signal output from the sensor 51, the control unit 111 measures a blood flow state of a user such as a pulse rate, a pulse fluctuation, and a blood flow volume. For example, the control unit 111 executes a process for detecting, by using infrared ray, change in flow volume of a red blood cell flowing through a blood vessel. The control unit 111 executes a process for detecting a frequency at which a Doppler shift caused by a red blood cell flowing through a blood vessel is generated. In other words, the control unit 111 executes a process for analyzing a signal related to irradiated light and/or reflected light so as to measure a blood flow state. The blood flow state to be measured is not limited to a pulse rate, a pulse fluctuation, a blood flow volume of a user, or the like; and it is sufficient that the state is related to blood flow of a user.

The communication unit 113 transmits and receives information to and from the display 120. Note that the controller 110 and the display 120 may be connected by using wireless communication, or may be connected by using wired communication. The control unit 111 outputs information indicating a measured blood flow state to the display 120 via the communication unit 113.

The display 120 displays information indicating a measured blood flow state. Thus, a user is able to recognize (grasp) his/her blood flow state. The display 120 may be a display of a remote controller that is configured to output an operation instruction of the toilet-seat device 20 or a washing instruction of the flush toilet 10, or may be a display of a portable terminal used by a user; however, this is merely one example, and not limited thereto.

The sensor 51 is not limited to the above-mentioned sensor that is configured to measure a blood flow state of a user, and may be another-type sensor such as a load sensor that is configured to measure a body weight of a user, and a body fat sensor that is configured to measure a body fat ratio of a user.

Returning to explanation of FIG. 1, an opening 32 is formed in the seating surface 31 of the toilet seat 30. The sensor unit 40 according to the embodiment is fixed to the toilet seat 30 such that a part thereof is exposed from the above-mentioned opening 32. In a case where a user sits on the toilet seat 30 to which the sensor unit 40 is fixed in the above-mentioned manner, the toilet seat 30 is deformed in some cases due to a body weight of the user. A sensor substrate on which the sensor 51 is mounted as described later is housed in the sensor unit 40, and thus in a case where deformation occurs in the toilet seat 30, there presents possibility that load works on the sensor substrate due to the deformation.

Thus, in the present embodiment, even in a case where the toilet seat 30 is deformed due to sitting of a user, load working on the sensor substrate is to be reduced.

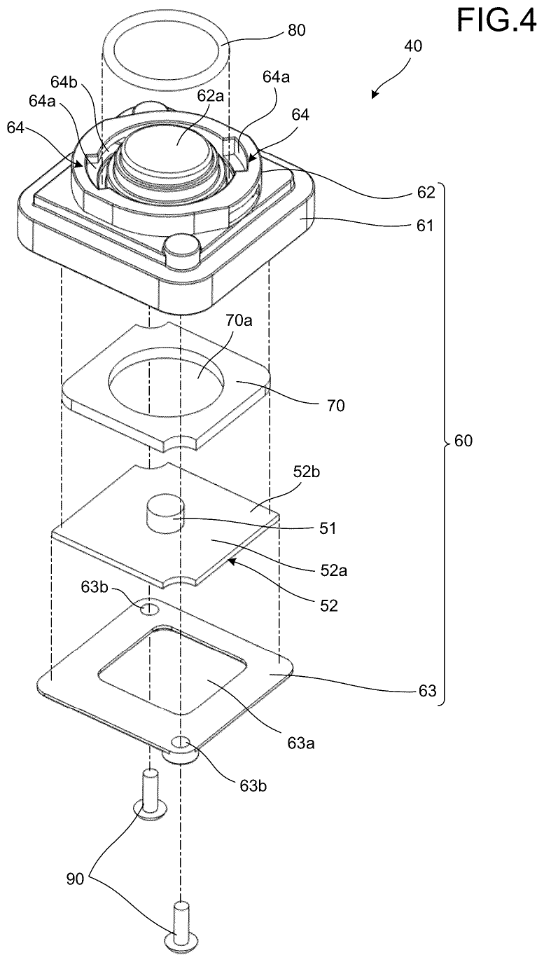

Details of the sensor unit 40 according to the present embodiment will be explained with reference to FIG. 3 and FIG. 4. FIG. 3 is a perspective view illustrating the sensor unit 40. FIG. 4 is an exploded perspective view illustrating the sensor unit 40.

The sensor unit 40 includes a sensor substrate 52 (not appearing in FIG. 3), a sensor case 60, a first elastic member 70 (not appearing in FIG. 3), and a second elastic member 80. Note that the sensor unit 40 is one example of a sensor unit.

As illustrated in FIG. 4, the sensor 51 is mounted on the sensor substrate 52. For example, the sensor substrate 52 is made of resin, and an upper surface thereof is a principal surface 52a. The sensor 51 is mounted in the vicinity of the center of the principal surface 52a of the sensor substrate 52. Note that illustration thereof is omitted, the sensor 51 includes an irradiation unit that is configured to irradiate irradiation light and/or a light receiving unit that is configured to receive reflected light.

The sensor case 60 houses therein the sensor substrate 52 and the first elastic member 70. The sensor case 60 is made of resin; however, not limited thereto.

The sensor case 60 includes a main body part 61, a protruding part 62, and a pressing member 63. The main body part 61 is hollowly formed in rectangular parallelepiped, for example. A lower side of the main body part 61 is opened.

The protruding part 62 is formed to protrude upward from an upper part of the main body part 61. The protruding part 62 is hollowly formed in cylindrical-shaped, for example. A sensor window 62a is provided in an upper part of the protruding part 62, which transmits irradiated light and reflected light. In other words, the sensor 51 irradiates irradiated light toward a user via the sensor window 62a, and further receives reflected light that is reflected from a blood vessel of a user via the sensor window 62a.

Engaging parts 64 for engaging and fixing the sensor case 60 with and to the toilet seat 30 are formed in the protruding part 62. Each of the engaging parts 64 includes an insertion hole 64a whose upper portion is opened, and a groove 64b that extends from the insertion hole 64a along a circumferential direction of the protruding part 62.

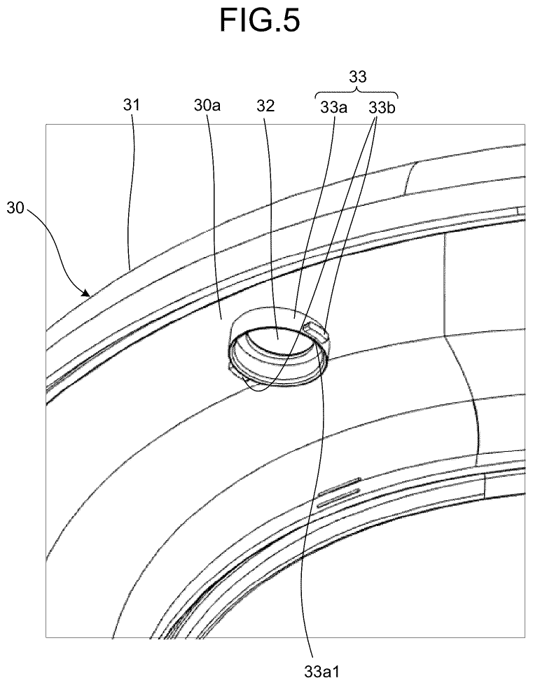

Herein, before continuing explanation of the engaging part 64, a configuration of the toilet seat 30, which is corresponding to the engaging part 64, will be explained with reference to FIG. 5. FIG. 5 is a perspective view illustrating the toilet seat 30 when viewed from the underneath. Note that in FIG. 5, for convenience of explanation, illustration of a bottom surface part (lower surface part) of the toilet seat 30 which is opposite to the seating surface 31 is omitted.

As illustrated in FIG. 5, the toilet seat 30 includes an engaged part 33 that is engaged with the engaging part 64 of the sensor case 60. The engaged part 33 is formed on a side of an inner surface 30a of the hollow toilet seat 30, and near the opening 32 of the toilet seat 30. The engaged part 33 includes a cylinder part 33a and claw parts 33b.

The cylinder part 33a is formed so as to protrude downward while surrounding the opening 32. Each of the claw parts 33b is formed so as to protrude from a side of a lower end 33a1 of the cylinder part 33a toward an outer side in a radial direction of the cylinder part 33a. The plurality of claw parts (for example, two) 33b is formed in the cylinder part 33a. Note that arrangement positions and the number of the claw parts 33b are merely examples, and not limited thereto.

The engaging part 64 illustrated in FIG. 4 and the like is engaged with the engaged part 33 configured as described above, and thus the sensor case 60 is fixed to the toilet seat 30. Specifically, first of all, the claw parts 33b of the engaged part 33 are inserted into the insertion holes 64a of the engaging part 64. Next, in a case where the sensor case 60 is rotated along a circumferential direction of the protruding part 62, the claw parts 33b of the engaged part 33 are slid along the grooves 64b of the engaging part 64 to be engaged therewith. Thus, the engaging part 64 is engaged with the engaged part 33, and further the sensor case 60 is fixed to the toilet seat 30. In other words, the main body part 61, the protruding part 62, and the like of the sensor case 60 are fixed to the toilet seat 30.

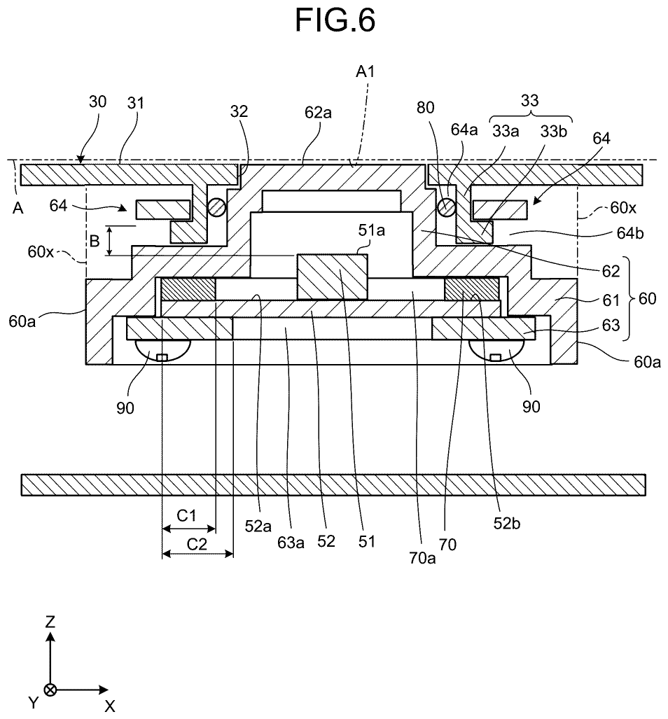

The state where the sensor case 60 is fixed to the toilet seat 30 will be explained with reference to FIG. 6. FIG. 6 is a cross sectional view taken along a line VI-VI illustrated in FIG. 1. Note that in FIG. 6, a user A sitting on the seating surface 31 of the toilet seat 30 is indicated by using a two-dot chain line, and further a measurement part of the user A is provided with a reference symbol “A1”.

As illustrated in FIG. 6, in a state where the sensor case 60 is fixed to the toilet seat 30, the sensor window 62a of the protruding part 62 is located so as to expose from the opening 32 of the toilet seat 30. Note that in the sensor case 60, details such as positions where the engaging parts 64 are arranged will be mentioned later.

Continuing explanation of FIG. 4 and FIG. 6, the pressing member 63 is formed in plate-shaped. The pressing member 63 is attached to the main body part 61 from a side of an opening in a lower surface of the main body part 61 to be fixed to the main body part 61. In this case, the sensor substrate 52 and the first elastic member 70 are inserted between the main body part 61 and the pressing member 63.

Specifically, a plurality of screw insertion

holes 63b (for example, two) is formed in the pressing member 63. Screw holes (not appearing in FIG. 4 and the like) are formed in positions corresponding to the screw insertion holes 63b in the main body part 61. For example, in a state where the sensor substrate 52 and the first elastic member 70 are arranged between an upper surface of the pressing member 63 and the main body part 61, screws 90 are inserted into the screw insertion holes 63b and further are fixed to screw holes of the main body part 61 so that the pressing member 63 is fixed to the main body part 61.

The sensor substrate 52 is inserted between the main body part 61 and the pressing member 63 having been fixed to the main body part 61. In other words, the sensor substrate 52 is fixed to the pressing member 63.

A non-contact part 63a is formed in the pressing member 63, which is not in contact with the sensor substrate 52. The non-contact part 63a is an opening part (notched part) that is formed near the center of the plate-shaped pressing member 63.

The first elastic member 70 is made of material having elasticity, such as rubber. A packing or the like may be used as the first elastic member 70. In a state where the pressing member 63 is fixed to the main body part 61, the first elastic member 70 is arranged (namely, inserted) between the sensor substrate 52 and the main body part 61. Note that the first elastic member 70 is one example of a substrate-side elastic member.

The first elastic member 70 is formed in plate-shaped, and an opening 70a is formed near the center thereof. The above-mentioned opening 70a is formed in a position corresponding to the sensor 51 of the sensor substrate 52. Specifically, in a state where the first elastic member 70 is arranged between the sensor substrate 52 and the main body part 61, the opening 70a is formed in a position such that the sensor 51 is inserted into the opening 70a. Caused by the opening 70a, the first elastic member 70 is formed so as to cover a part 52b that is other than the sensor 51 mounted on the sensor substrate 52.

The second elastic member 80 is made of material having elasticity, such as rubber. An O-ring may be used as the second elastic member 80. As illustrated in FIG. 6, the second elastic member 80 is arranged (namely, inserted) between the protruding part 62 of the sensor case 60 and the toilet seat 30 (precisely, cylinder part 33a of engaged part 33). Note that the second elastic member 80 is one example of a protruding-part-side elastic member.

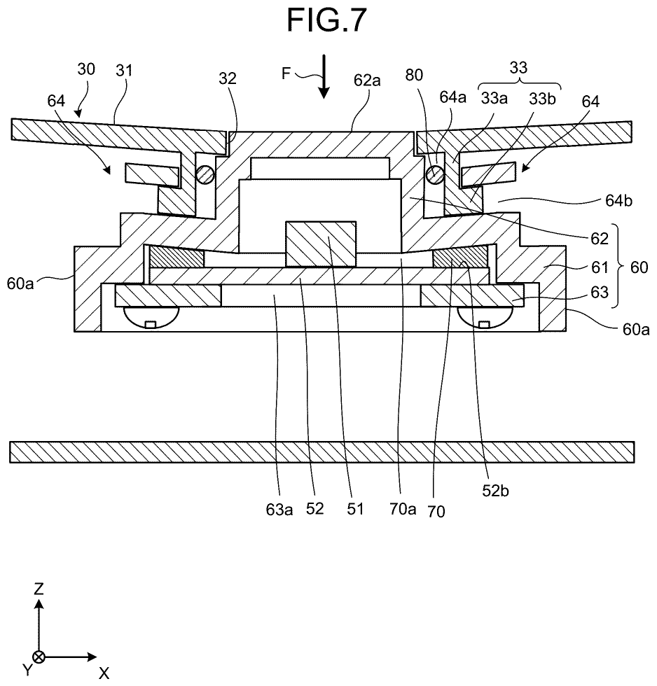

The sensor unit 40 according to the present embodiment is configured as described above, so that it is possible to reduce load working on the sensor substrate 52 even in a case where the toilet seat 30 is deformed due to sitting of a user. This point will be explained with reference to FIG. 7. FIG. 7 is a diagram illustrating a state where the toilet seat 30 is deformed due to sitting of a user, and further is a cross sectional view that is similar to FIG. 6.

As illustrated in FIG. 7, in a case where a user sits on the toilet seat 30 and thus a load F works, deformation occurs in the toilet seat 30. Due to the above-mentioned deformation, load works on the protruding part 62 and the main body part 61 of the sensor case 60 from the engaged part 33 via the engaging part 64, and thus the main body part 61 is also deformed in some cases.

As described above, in a case where the toilet seat 30 and/or the main body part 61 is deformed due to sitting of a user, the sensor substrate 52 is fixed to the pressing member 63, and thus it is possible to reduce load that works on the sensor substrate 52. In other words, the sensor case 60 includes the main body part 61 that is fixed to the toilet seat 30 and the pressing member 63 that is not directly fixed to the toilet seat 30, and the sensor substrate 52 is fixed to the above-mentioned pressing member 63. Thus, it is possible to reduce load that works on the sensor substrate 52 even in a case where the toilet seat 30 and the like are deformed.

Moreover, the pressing member 63 includes the non-contact part 63a that is not in contact with the sensor substrate 52. In other words, the pressing member 63 and the sensor substrate 52 are configured to be in partially contact with each other. Thus, for example, even in a case where the pressing member 63 is deformed to largely bend due to deformation of the toilet seat 30, it is possible to reduce load working on the sensor substrate 52 by amount corresponding to non-contact portion with the sensor substrate 52.

Herein, with reference to FIG. 6 in a side view, a main body part side length C1 by which the main body part 61 is in contact with the sensor substrate 52, and a pressing member side length C2 by which the pressing member 63 is in contact with the sensor substrate 52 will be explained. Note that the first elastic member 70 is arranged between the main body part 61 and the sensor substrate 52. Thus, the main body part side length C1 is a length by which the main body part 61 is in contact with the sensor substrate 52 via the first elastic member 70.

As illustrated in FIG. 6, the main body part side length C1 is set to be shorter than the pressing member side length C2 (namely, C1<C2). In other words, the pressing member side length C2 is set to be longer than the main body part side length C1.

As described above, the main body part side length C1 is set to be shorter than the pressing member side length C2, so that it is possible to more reduce load working on the sensor substrate 52 from a side of the main body part 61 when the toilet seat 30 is deformed than a case where the main body part side length C1 is longer than the pressing member side length C2. In other words, in a case where the main body part side length C1 is longer than the pressing member side length C2, load working on the sensor substrate 52 from a side of the main body part 61 increases in accordance with the principle of leverage; however, the main body part side length C1 is set to be shorter than the pressing member side length C2 as described above, so that it is possible to reduce load that works on the sensor substrate 52.

Furthermore, the first elastic member 70 is arranged between the sensor substrate 52 and the main body part 61. Thus, as illustrated in FIG. 7, for example, even in a case where deformation occurs in the toilet seat 30 and the main body part 61 is accordingly deformed, the first elastic member 70 receives and absorbs the deformation of the main body part 61, so that it is possible to reduce load that works on the sensor substrate 52.

The first elastic member 70 is formed so as to cover the part 52b other than the sensor 51 mounted on the sensor substrate 52. The first elastic member 70 includes the above-mentioned opening 70a, and thus is formed so as to cover the part 52b other than a part, on which the sensor 51 is mounted, of the principal surface 52a of the sensor substrate 52. Thus, for example, even in a case where deformation occurs in the toilet seat 30 and the main body part 61 due to sitting of a user, and the first elastic member 70 affected by the deformation of the main body part 61 is deformed, it is possible to reduce effects working on the sensor 51 as much as possible. Therefore, it is possible to prevent reduction in detection accuracy of the sensor 51.

Next, details of a position will be explained, in which the engaging part 64 for fixing the sensor case 60 to the toilet seat 30 is arranged.

As illustrated in FIG. 6 and FIG. 7, each of the engaging parts 64 is arranged inner than an outer edge 60a of the sensor case 60. Specifically, the engaging parts 64 are arranged inner than a virtual line 60x (see FIG. 6) that extends upward along the outer edge 60a from the outer edge 60a of the sensor case 60.

Thus, even in a case where deformation occurs in the toilet seat 30 due to sitting of a user, it is possible to reduce load that works on the sensor substrate 52. In other words, as illustrated in FIG. 7, even in a case where deformation occurs in the toilet seat 30, the engaging parts 64 follow the deformation of the toilet seat 30 to receive a deformation force, so that it is possible to reduce deformation of the sensor case 60 itself. Moreover, the engaging parts 64 are arranged inner than the outer edge 60a of the sensor case 60, and thus application of deformation due to the following of the engaging parts 64 to whole of the sensor case 60 can be reduced, so that it is possible to reduce load that works on the sensor substrate 52. Additionally, the sensor case 60 is fixed to the toilet seat 30 by the engaging parts 64, and thus even in a case where a user erroneously pushes an exposed part of the sensor case 60, for example, it is possible to prevent a case where the sensor case 60 comes off the toilet seat 30.

The sensor case 60 is exposed from the opening 32 that is formed in the seating surface 31 of the toilet seat 30. Next, the engaging parts 64 arranged upper than the sensor substrate 52, preferably, upper than the sensor 51 that is mounted on the sensor substrate 52. Specifically, a center position of the engaging parts 64 in the up-and-down direction is arranged upper than the principal surface (in other words, upper surface) 52a on an upper side of the sensor substrate 52, preferably, is arranged upper than an upper surface 51a of the sensor 51 by a distance (height) B. Note that the distance B can be set to an arbitrary value.

As described above, the engaging parts 64 and the sensor 51 are arranged to separate from each other in a height direction (namely, up-and-down direction), so that it is possible to more reliably reduce load that works on the sensor substrate 52 due to deformation of the engaging parts 64.

Moreover, the second elastic member 80 is arranged between the protruding part 62 and the toilet seat 30. Thus, even in a case where the toilet seat 30 is deformed, it is possible to more reduce load that works on the sensor substrate 52. In other words, in the engaging parts 64, there presents possibility that a play is generated due to a size error and the like, and an effect of load applied from the toilet seat 30 gathers in some part of the engaging parts 64. Thus, the second elastic member 80 is arranged between the toilet seat 30 and the protruding part 62 of the sensor case 60, so that it is possible to regulate movement of the sensor case 60 with respect to the toilet seat 30 in the height direction while reducing a play due to a size error and the like. Therefore, an effect of load applied from the toilet seat 30 hardly gathers in some part of the engaging parts 64, so that it is possible to more reliably reduce load that works on the sensor substrate 52, as a result. Moreover, because of the second elastic member 80, it is possible to prevent water from penetrating into the toilet seat 30, and further to absorb an impact to the sensor substrate 52 caused by opening/closing of the toilet seat 30 or sitting.

The second elastic member 80 is arranged such that a height thereof in the up-and-down direction is equal to heights of the engaging parts 64. Note that the equal height is not necessarily be a strictly equal height, and it is sufficient that heights are appropriately the same within a permissible range having some extent.

Thus, even in a case where deformation occurs in the toilet seat 30 due to sitting of a user, both of the engaging parts 64 and the second elastic member 80 are capable of receiving the deformation while following the deformation of the toilet seat 30. Therefore, it is possible to more reduce load that works on the sensor substrate 52.

A toilet-seat device according to one aspect of

the embodiment includes: a hollow toilet seat; and a sensor unit including a sensor case that is fixed to be exposed from an opening formed in the toilet seat, the sensor case housing therein a sensor substrate, wherein the sensor case includes an engaging part that is engaged with the toilet seat to fix the sensor case to the toilet seat, and the engaging part is arranged in a position that is inner than an outer edge of the sensor case.

Thus, even in a case where deformation occurs in a toilet seat due to sitting of a user, it is possible to reduce load that works on a sensor substrate. In other words, even in a case where deformation occurs in a toilet seat, an engaging part follows the deformation of the toilet seat so as to receive a deformation force, so that deformation hardly occurs in a sensor case itself. Moreover, an engaging part is arranged in a position inner than an outer edge of the sensor case, so that it is possible to reduce a case where deformation due to following of the engaging part is applied to whole of the sensor case, and further to reduce load that works on the sensor substrate. Furthermore, the sensor case is fixed to the toilet seat by the engaging part, even in a case where a user erroneously pushes an exposed part of the sensor case, for example, it is possible to prevent the sensor case coming off the toilet seat.

The sensor case is exposed from the opening that is formed in a seating surface of the toilet seat, and the engaging part is arranged in a position that is upper than the sensor substrate.

As described above, the engaging part and the sensor are arranged such that heights thereof are separated from each other in the height direction, so that it is possible to reduce load that works on the sensor substrate due to deformation of the engaging part.

The sensor case includes: a main body part; and a protruding part that protrudes from the main body part to be exposed from the opening of the toilet seat, and a protruding-part-side elastic member is arranged between the protruding part and the toilet seat.

Thus, even in a caser where deformation occurs in the toilet seat, it is possible to more reduce load that works on the sensor substrate. In other words, in the engaging part, there presents possibility that a play occurs caused due to a size error or the like, an effect of load applied from the toilet seat gathers in a part of the engaging part. Thus, the protruding-part-side elastic member is arranged between the toilet seat and the protruding part of the sensor case, so that it is possible to regulate movement of the sensor case with respect to the toilet seat in the height direction while reducing a play caused by a size error and the like. Therefore, an effect of load applied from the toilet seat hardly gathers in some part of the engaging part, so that it is possible to more reliably reduce load that works on the sensor substrate, as a result. Moreover, because of the protruding-part-side elastic member, it is possible to prevent water from penetrating into the toilet seat, and further to absorb an impact to the sensor substrate due to opening/closing of the toilet seat or sitting.

The protruding-part-side elastic member is arranged such that a height of the protruding-part-side elastic member in an up-and-down direction is substantially equal to a height of the engaging part in the up-and-down direction.

Thus, even in a case where deformation occurs in the toilet seat due to sitting of a user, both of the engaging part and the protruding-part-side elastic member are capable of receiving the deformation while following the deformation of the toilet seat. Therefore, it is possible to more reduce load that works on the sensor substrate.

The sensor case includes: a main body part that is fixed to the toilet seat; and a pressing member that is fixed to the main body part, wherein the sensor substrate is fixed to the pressing member.

Thus, in a case where the toilet seat and/or the main body part is deformed due to sitting of a user, the sensor substrate is fixed to the pressing member, and thus it is possible to reduce load that works on the sensor substrate. In other words, the sensor case includes the main body part that is fixed to the toilet seat and the pressing member that is not directly fixed to the toilet seat, and the sensor substrate is fixed to the above-mentioned pressing member. Thus, it is possible to reduce load that works on the sensor substrate even in a case where the toilet seat and the like are deformed.

The pressing member includes: a non-contact part that is not in contact with the sensor substrate.

As described above, the pressing member includes the non-contact part, so that the pressing member and the sensor substrate are configured to be in partially contact with each other. Thus, for example, even in a case where the pressing member is deformed to largely bend due to deformation of the toilet seat, it is possible to reduce load working on the sensor substrate by amount corresponding to non-contact portion with the sensor substrate.

The sensor substrate is arranged between the pressing member and the main body part to be fixed, and in a side view, a main body part side length by which the main body part is in contact with the sensor substrate is set to be shorter than a pressing member side length by which the pressing member is in contact with the sensor substrate.

As described above, the main body part side length is set to be shorter than the pressing member side length, so that it is possible to more reduce load working on the sensor substrate from a side of the main body part when the toilet seat is deformed than a case where the main body part side length is longer than the pressing member side length. In other words, in a case where the main body part side length is longer than the pressing member side length, load working on the sensor substrate from a side of the main body part increases in accordance with the principle of leverage; however, the main body part side length is set to be shorter than the pressing member side length as described above, it is possible to reduce load that works on the sensor substrate.

A substrate-side elastic member, which is arranged between the sensor substrate and the main body part, is included.

Even in a case where deformation occurs in the toilet seat and the main body part is accordingly deformed, the first elastic member receives and absorbs the deformation of the main body part, so that it is possible to reduce load that works on the sensor substrate.

The substrate-side elastic member is formed to cover a part other than a sensor that is mounted on the sensor substrate.

Thus, for example, even in a case where deformation occurs in the toilet seat and the main body part due to sitting of a user, and the substrate-side elastic member affected by the deformation of the main body part is deformed, it is possible to reduce effects working on the sensor as much as possible. Therefore, it is possible to prevent reduction in detection accuracy of the sensor.

According to one aspect of the embodiment, even in a where deformation occurs in a toilet seat due to sitting of a user, it is possible to reduce load that works on a sensor substrate.

### Supplementary Note

(1)

A toilet-seat device including:
a hollow toilet seat; and
a sensor unit including a sensor case that is fixed to be exposed from an opening formed in the toilet seat, the sensor case housing therein a sensor substrate, wherein
the sensor case includes an engaging part that is engaged with the toilet seat to fix the sensor case to the toilet seat, and
the engaging part is arranged in a position that is inner than an outer edge of the sensor case.
   (2)

The toilet-seat device according to (1), wherein
the sensor case is exposed from the opening that is formed in a seating surface of the toilet seat, and
the engaging part is arranged in a position that is upper than the sensor substrate.

(3)

The toilet-seat device according to (1) or (2), wherein
the sensor case includes:
   a main body part; and
   a protruding part that protrudes from the main body part to be exposed from the opening of the toilet seat, and
a protruding-part-side elastic member is arranged between the protruding part and the toilet seat.
   (4)

The toilet-seat device according to (3), wherein
the protruding-part-side elastic member is arranged such that a height of the protruding-part-side elastic member in an up-and-down direction is substantially equal to a height of the engaging part in the up-and-down direction.
   (5)

The toilet-seat device according to (1) to (4), wherein
the sensor case includes:
   a main body part that is fixed to the toilet seat; and
   a pressing member that is fixed to the main body part, and
the sensor substrate is fixed to the pressing member.
   (6)

The toilet-seat device according to (5), wherein
the pressing member includes:
   a non-contact part that is not in contact with the sensor substrate.
      (7)

The toilet-seat device according to (5) or (6), wherein
the sensor substrate is arranged between the pressing member and the main body part to be fixed, and
in a side view, a main body part side length by which the main body part is in contact with the sensor substrate is set to be shorter than a pressing member side length by which the pressing member is in contact with the sensor substrate.
   (8)

The toilet-seat device according to (5) to (7), further including:
a substrate-side elastic member that is arranged between the sensor substrate and the main body part.
   (9)

The toilet-seat device according to (8), wherein
the substrate-side elastic member is formed to cover a part other than a sensor that is mounted on the sensor substrate.

Although the invention has been described with respect to specific embodiments for a complete and clear disclosure, the appended claims are not to be thus limited but are to be construed as embodying all modifications and alternative constructions that may occur to one skilled in the art that fairly fall within the basic teaching herein set forth.

What is claimed is:

1. A toilet-seat device comprising:
a hollow toilet seat; and
a sensor unit including a sensor case that is fixed to be exposed from an opening formed in the toilet seat, the sensor case housing therein a sensor substrate, wherein
the sensor case includes an engaging part that is engaged with the toilet seat to fix the sensor case to the toilet seat, and
the engaging part is arranged in a position that is inner than an outer edge of the sensor case.

2. The toilet-seat device according to claim 1, wherein
the sensor case is exposed from the opening that is formed in a seating surface of the toilet seat, and
the engaging part is arranged in a position that is upper than the sensor substrate.

3. The toilet-seat device according to claim 1, wherein
the sensor case includes:
a main body part; and
a protruding part that protrudes from the main body part to be exposed from the opening of the toilet seat, and
a protruding-part-side elastic member is arranged between the protruding part and the toilet seat.

4. The toilet-seat device according to claim 3, wherein
the protruding-part-side elastic member is arranged such that a height of the protruding-part-side elastic member in an up-and-down direction is substantially equal to a height of the engaging part in the up-and-down direction.

5. The toilet-seat device according to claim 1, wherein
the sensor case includes:
a main body part that is fixed to the toilet seat; and
a pressing member that is fixed to the main body part, and
the sensor substrate is fixed to the pressing member.

6. The toilet-seat device according to claim 5, wherein
the pressing member includes:
a non-contact part that is not in contact with the sensor substrate.

7. The toilet-seat device according to claim 5, wherein
the sensor substrate is arranged between the pressing member and the main body part to be fixed, and
in a side view, a main body part side length by which the main body part is in contact with the sensor substrate is set to be shorter than a pressing member side length by which the pressing member is in contact with the sensor substrate.

8. The toilet-seat device according to claim 5, further comprising:
a substrate-side elastic member that is arranged between the sensor substrate and the main body part.

9. The toilet-seat device according to claim 8, wherein
the substrate-side elastic member is formed to cover a part other than a sensor that is mounted on the sensor substrate.
